(19) United States
(12) Patent Application Publication
(10) Pub. No.: US 2017/0196593 A1
(43) Pub. Date: Jul. 13, 2017

(54) DEVICE AND METHODS FOR MANIPULATING A UTERUS OR OTHER BODILY TISSUE

(71) Applicant: Bioceptive, Inc., New Orleans, LA (US)

(72) Inventors: Shuchi P. KHURANA, Metairie, LA (US); Benjamin D. CAPPIELLO, New Orleans, LA (US); Colin A. DAVIS, New Orleans, LA (US); Charles R. DEARK, III, Mandeville, LA (US)

(73) Assignee: Bioceptive, Inc., New Orleans, LA (US)

(21) Appl. No.: 15/418,285

(22) Filed: Jan. 27, 2017

Related U.S. Application Data
(63) Continuation of application No. PCT/US2015/042523, filed on Jul. 28, 2015.
(60) Provisional application No. 62/029,820, filed on Jul. 28, 2014.

(51) Int. Cl. A61B 17/42; A61F 6/18; B65D 81/18; A61M 1/00

(52) U.S. Cl. CPC A61B 17/4241; A61M 1/0056; A61F 6/18; B65D 81/18; A61B 2017/00473

(57) ABSTRACT

An apparatus is described that includes a vacuum line and a head coupled to a first end portion of the vacuum line. The vacuum line defines a fluid passageway. The head is configured to be removably coupled to a first portion of a reusable device. The head includes a contact portion configured to contact a surface associated with a target location. The contact portion is configured to circumscribe an opening to a bodily cavity associated with the target location. The head defines a volume through which a vacuum is conveyed to the surface associated with the target location. The volume is in fluid communication with the fluid passageway of the vacuum line.

### CROSS-REFERENCE TO RELATED APPLICATIONS

This application is a continuation of International Application No. PCT/US2015/042523, entitled “Device and Methods for Manipulating a Uterus or Other Bodily Tissue,” filed Jul. 28, 2015, which claims priority to and the benefit of U.S. Provisional Patent Application Ser. No. 62/029,820 entitled “Device and Methods for Manipulating a Uterus or other Bodily Tissue for Implanting a Device or Administering a Drug,” filed Jul. 28, 2014, the disclosures of each of which are incorporated herein by reference in their entirety.

### BACKGROUND

The embodiments described herein relate generally to devices and methods for attaching a medical device to a uterus or any other bodily tissue. The attached medical device can be used to produce a pathway for another medical device, such as an inserter or probe for navigating a bodily passageway, for implanting a medical device or administering a drug. Specifically, the embodiments described herein relate to an apparatus for manipulating bodily tissue and/or inserting a device into a bodily passageway, such as the cervical canal, while limiting trauma to the surrounding tissue during insertion. The embodiments described herein also facilitate reducing pressure on the bodily tissue when detecting tissue with the distal tip of the apparatus.

Current procedures that require entry into the endometrium may require the cervix to be stabilized using a cervical tenaculum and that the cervix be dilated using a cervical dilator or Os finder, adding an additional step to these procedures. The tenaculum can be painful and causes bleeding in many cases, which can add procedural time for blood management. This dilation allows instruments to enter the cervix. These dilations are performed for many procedures involving the uterus, such as dilation and curettage, manual vacuum aspiration, electric vacuum aspiration, endometrial biopsy, dilation and evacuation, gynecological brachytherapy, insertion of various contraceptive devices, and certain abortion procedures, among others. These dilations are performed to ease passage of instruments though the cervical canal and prevent damage to the tissue during insertion. Damage can be caused by the physical act of insertion as the distal tip of the insertion member can scrape or catch on surrounding cervical tissue. In many cases, the health care provider may choose to forego the use of a cervical dilator in belief that certain devices can perform the similar function of dilating the cervical canal and also creating an established passageway through which the other instruments will enter. This is a dangerous part of some procedures where many perforations (creation of false passageways) can occur.

Thus, a need exists for improved cervical manipulation devices and insertion facilitation devices that can prevent the trauma caused from engagement of and insertion into the uterus without the use of painful and/or potentially dangerous instruments, such as a tenaculum, and procedures, such as cervical dilation, to facilitate insertion. Creation of a reusable version of the above mentioned devices could further reduce the cost of these procedures while maintaining or improving the benefits of the new improved insertion facilitation device.

### SUMMARY

The embodiments described herein include variations of a medical device such that some parts of the device can be reused while other parts of the device are disposed of after one use or after a limited number of uses. Various embodiments of an insertion member may include any of the following or any combination: the vacuum port is disposable and connects to a housing that has a one-way filter for bodily fluids. The housing may be a part of the vacuum port. The reservoir assembly connects to the reusable portion of the device where the connection is designed as a quick snap feature or any variation thereof that can quickly engage or disengage the reusable part with the disposable part of the device. The reusable part of the device is designed such that internal components are sealed to occlude any liquid from entering the inside during disinfection stage.

### BRIEF DESCRIPTION OF THE DRAWINGS

FIG. 1 is an illustration of a portion of the female reproductive system provided for reference.

FIGS. 2 and 3 are schematic illustrations of a portion of a delivery device according to an embodiment, in a coupled and a decoupled configuration, respectfully.

FIGS. 4 and 5 are a front perspective view and a rear perspective view, respectively, of a portion of a medical device according to an embodiment.

FIGS. 6 and 7 are a front perspective view and a rear perspective view, respectively, of a vacuum head included in the disposable assembly of FIGS. 4 and 5.

FIG. 8 is a cross-sectional view of the vacuum head of FIG. 6 taken along the line 12-12 in FIG. 6.

FIG. 9 is a cross-sectional view of FIG. 8 illustrating the vacuum head in contact with a portion of the uterus.

FIG. 10 is the cross-sectional view of a vacuum head of a disposable assembly according to an embodiment.

FIG. 11 is a flowchart illustrating a method for inserting an instrument into a bodily cavity using a disposable tissue manipulation device and a reusable medical device.

FIG. 12 is a partially exploded view of a medical device containing a reusable medical device and disposable medical device, according to an embodiment.

FIG. 13 is front perspective view of the medical device of FIG. 12.

FIG. 14 is a top view of the vacuum indicator on the handle of the medical device shown in FIG. 12.

FIG. 15 is a perspective view of the reservoir assembly shown in FIG. 12, including an attachment member.

FIG. 16 is a perspective view of the reservoir assembly shown in FIG. 12, including an attachment member and a vacuum line.

FIG. 17 is a perspective view of a connection member of the medical device of FIG. 12.

FIG. 18 is a side perspective view of a resetting instrument for use with an insertion device, according to an embodiment.

### DETAILED DESCRIPTION

In some embodiments, a delivery device and/or tissue manipulation device of the types described herein can facilitate an intrauterine procedure. The embodiments described herein can reduce the risk of complications due to poor insertion technique and can increase the ease of insertion of, for example, an intrauterine device (IUD). The devices shown and described herein can also be used to insert any another device, implant and/or pharmaceutical into a bodily passage, such as, for example, the female reproductive system. In some embodiments, the devices and methods described herein can be used for insertion of a catheter, enema, drug delivery object, imaging tools, endoscopy, tubes (e.g., into the lungs and other body cavities), or other applications where precise insertion would be beneficial to the efficacy of the treatment and/or to reduce complications or pain. Furthermore, the devices and methods described herein can provide gentler and/or easier approaches for navigating around and/or past obstacles or anatomical variations in bodily passageways, while also limiting trauma from excess pressure when detecting tissues with the distal tip of the insertion member.

In some embodiments, an insertion member can be configured to articulate with the cervix and can be used, for example, to insert an IUD into a woman's uterus with no other tools needed, and without the need for exceptional skill and/or training. The embodiments described herein focus, at least partially, on ease of use, repeatability, and precision of insertion. Thus, after a short training session, any health care provider can properly insert an IUD safely. The invention may also continue to be used with the additional tools currently used in known IUD insertions and other known procedures. For example, the device and methods described herein can be used with any of the tools and/or methods described in U.S. Patent Application Publication No. 2013/0291872 entitled, “Methods and Apparatus for Inserting a Device or Pharmaceutical Into a Body Cavity,” filed Apr. 16, 2013 (872 publication), PCT Publication No. W02013/082452 entitled, “Methods and Apparatus for Inserting a Device or Pharmaceutical Into a Uterus,” filed on Nov. 30, 2012 (452 PCT publication), and PCT Publication No. W02014/205351 entitled, “Devices and Methods for Manipulating Bodily Tissue,” filed on Jun. 20, 2014 ('351 PCT publication), the disclosures of which are incorporated herein by reference in their entireties.

In some embodiments, the devices described herein can be disposable, comprehensive devices that can be configured to articulate with a target tissue. As such, the devices can, for example, facilitate insertion of an IUD to a desired and/or predetermined position and/or orientation within the body.

In some embodiments, a delivery device can be configured to articulate with the cervix and can be used, for example, to insert an IUD into a woman's uterus with no other tools needed, and without the need for exceptional skill and/or training.

In some embodiments, a delivery device can include a vacuum line that defines a fluid passageway and has a first end portion and a second end portion. A head is coupled to the first end portion of the vacuum line. The head is configured to be removably coupled to a first portion of a reusable medical device. The head includes a contact portion configured to contact a surface associated with a target location (e.g., a cervix). The head defines a volume through which a vacuum can be conveyed to the surface associated with the target location. The volume is in fluid communication with the fluid passageway of the vacuum line.

In some embodiments, a method includes inserting a head of a disposable tissue manipulation device into a body until a contact portion of the head contacts a surface associated with a target location and a volume defined by the head at least partially circumscribes an opening to a bodily cavity associated with the target location. The disposable tissue manipulation device is removably coupled to a reusable medical device. The volume is fluidically coupled to a vacuum chamber of the reusable medical device. After inserting the head, an actuator at least partially disposed within the vacuum chamber of the reusable medical device is manipulated to produce a vacuum within the volume. At least a portion of the surface is drawn into the volume by the vacuum. After manipulating the actuator, the method includes inserting an instrument within a passageway defined by the head of the disposable tissue manipulation device until a distal end portion of the instrument is disposed within the bodily cavity. After inserting the instrument, the method includes decoupling the disposable tissue manipulation device from the reusable medical device.

In some embodiments, a kit includes a reusable medical device and a disposable assembly configured to be removably coupled to the reusable medical device. The reusable medical device defines a vacuum chamber and an actuator at least partially disposed within the vacuum chamber such that movement of the actuator within the vacuum chamber produces a vacuum. The disposable assembly includes a head and a reservoir coupled to the head. The head is configured to be fluidically coupled to the vacuum chamber, and defines a volume configured to at least partially circumscribe an opening to a bodily cavity associated with a target location. The head is configured to exert the vacuum on a surface of the target location via the volume. The reservoir is in fluid communication with the vacuum chamber of the reusable medical device when the reservoir is coupled to the reusable medical device. The reservoir includes a filter configured to (1) limit passage therethrough of particles having a size above a threshold value, and (2) allow air to pass therethrough sufficient to maintain the vacuum within the disposable assembly.

As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof.

As used herein, the words “proximal” and “distal” refer to direction closer to and away from, respectively, an operator of the medical device. Thus, for example, the end of the medicament delivery device contacting the patient's body would be the distal end of the medicament delivery device, while the end opposite the distal end would be the proximal end of the medicament delivery device.

The embodiments described herein can be formed or constructed of one or more biocompatible materials. Examples of suitable biocompatible materials include metals, glasses, ceramics, or polymers. Examples of suitable metals include pharmaceutical grade stainless steel, gold, titanium, nickel, iron, platinum, tin, chromium, copper, and alloys thereof. A suitable polymer may be biodegradable or non-biodegradable. Examples of suitable biodegradable polymers include polylactides, polyglycolides, polylactide-co-glycolides (PLGA), polyanhydrides, polyorthoesters, polyetheresters, polycaprolactones, polyesteramides, poly(butyric acid), poly(valeric acid), polyurethanes and copolymers and blends thereof. Examples of suitable non-biodegradable polymers include nylons, polyesters, polycarbonates, polyacrylates, polymers of ethylene-vinyl acetates and other acyl-substituted cellulose acetates, non-degradable polyurethanes, polystyrenes, polyvinyl chloride, polyvinyl fluoride, poly(vinyl imidazole), chlorosulphonate polyolefins, polyethylene oxide, blends and copolymers thereof. Moreover, the embodiments, described herein can be formed or constructed of one or more of the biocompatible materials and/or blends thereof based at least in part of a durometer of the constituent biocompatible material.

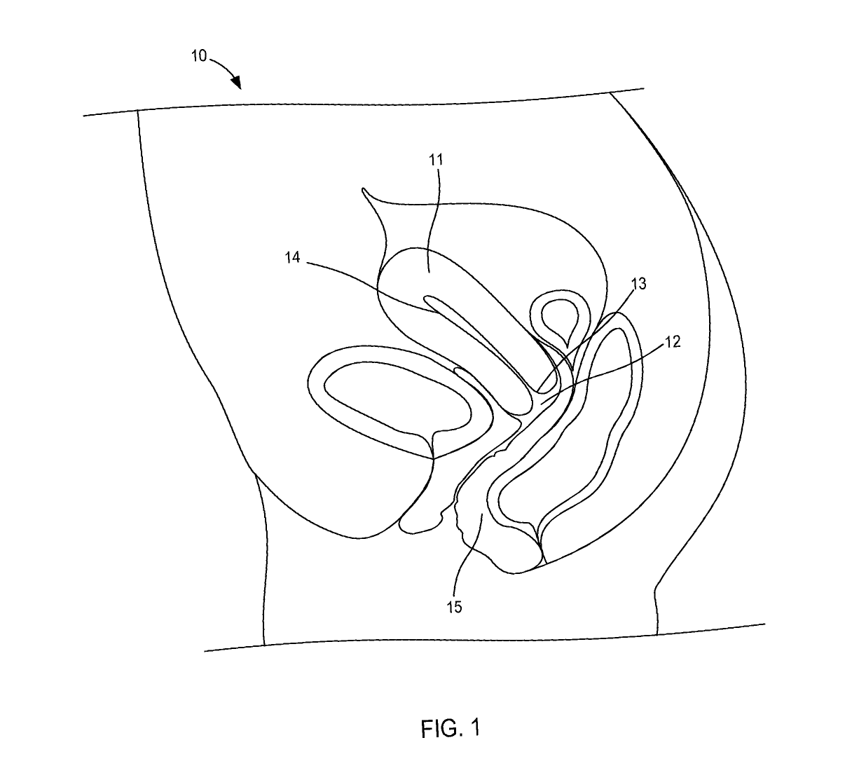

FIG. 1 is an illustration of a portion of the female reproductive system shown, for example, to provide context to the description of the devices and methods herein. That is to say, while specific portions of the female reproductive system are shown and described, it is not meant to be an exhaustive discussion of the female reproductive system. Rather, pertinent anatomical structures, passageways, etc. are presented by way of example to illustrate a use of the devices and methods described herein. While the female reproductive system is shown and described in FIG. 1, the devices and methods described herein can be used in other portions of the human body (e.g., either male or female). As shown in FIG. 1, a pelvic region of the female body 10 (also referred to as “body”) includes, inter alia, the vagina 15 and the uterus 11. More particularly, the uterus 11 is a substantially U-shaped or pearl-shaped and is positioned immediately posterior to the urinary bladder and in communication with the vaginal canal. The uterus 11 includes a neck portion known as the cervix 12, which defines a cervical os 13 providing access to an interior region of the uterus. Opposite the cervical os 13 (also referred to herein as “os”) is a portion of the uterus 11 known as the fundus 14. In some intrauterine procedures such as, for example, the insertion of an intrauterine device (IUD), it is desirable to deliver an implant through the vagina 15 and the os 13 to be implanted into a portion of the fundus 14. As is shown in FIG. 1, the insertion path is generally tortuous and often manipulation of a portion of the cervix 12 is used to allow access through the os 13, as described in further detail herein.

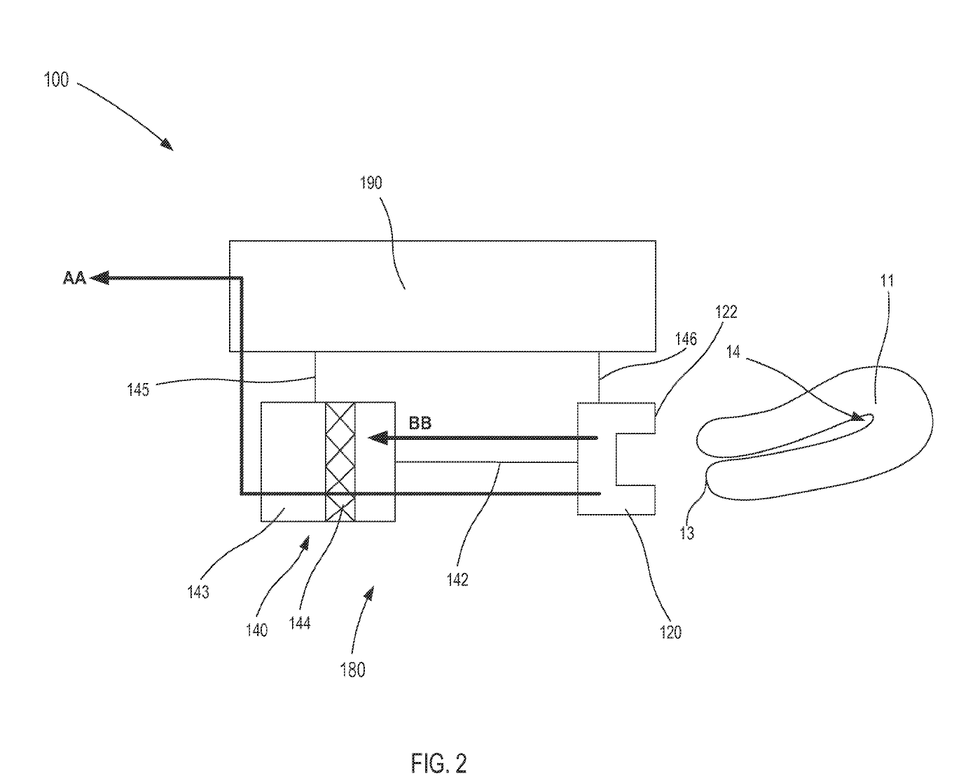

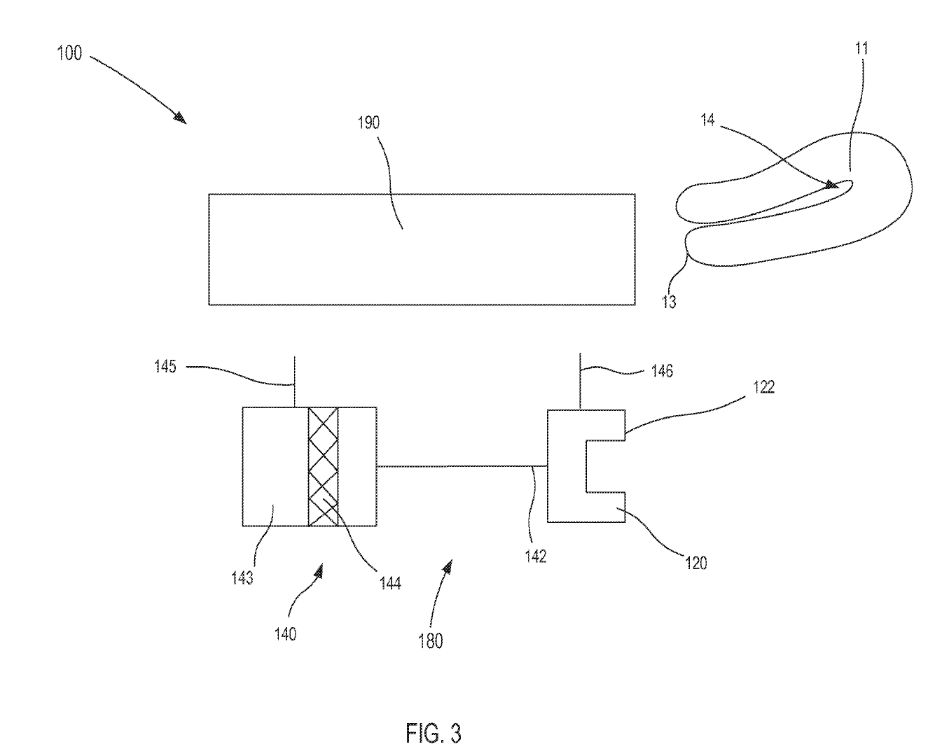

FIGS. 2 and 3 illustrate a medical device 100 in a coupled configuration and a decoupled configuration, respectively, according to an embodiment. The medical device 100 (and any of the other insertion devices and/or tissue engagement devices described herein) can be used with any of the sheaths, insertion members or the like described herein or in the '872 publication, the '452 PCT publication and/or the '351 PCT publication, which are incorporated by reference above. In some embodiments, the medical device 100 can be used to engage, manipulate and/or secure a bodily tissue to facilitate a procedure on the bodily tissue. The medical device 100 (also referred to herein as “device”) includes a reusable medical device (or portion) 190 and a disposable assembly 180. The disposable assembly 180 includes a head 120, a reservoir 140, and a vacuum line 142. The vacuum line 142 defines a fluid passageway have a first end portion coupled to the head 120 and a second end portion coupled to the reservoir 140. The disposable assembly 180 is configured to be removably coupled to the reusable medical device 190.

The head 120 can be removably coupled to a first portion of the reusable medical device 190 by a connection member 146. The connection member 146 is configured to mate with one or more corresponding connection members (not shown) of the reusable medical device 190 such that head 120 can be coupled to the reusable medical device 190 in any suitable position and/or orientation. For example, in some embodiments, the head 120 is fixedly coupled to the reusable medical device 190 such that a portion head 120 extends beyond an end surface of the reusable medical device 190 by a predetermined amount (e.g., that can be associated with an anatomical feature). In other embodiments, the head 120 is fixedly coupled to the reusable medical device 190 at a predetermined angle or orientation.

In yet other embodiments, the head 120 is movably coupled to the reusable medical device 190 such that the head 120 can move relative to the reusable medical device 190 before and/or during a procedure. For example, in some embodiments, the connection member 146 is configured such that the head 190 can rotate relative to the reusable medical device 190 with at least one degree of freedom. In this manner, the head 190 can be manipulated and/or moved during a procedure. In some embodiments, the connection member 146 of the head 120 can include a protrusion that is received within the connection member (not shown; including a corresponding opening) of the reusable medical device 190. The connection member 146 can include substantially cylindrical protrusions or the like that can be disposed in the corresponding openings defined by the reusable medical device to movably and removably couple the head 120 to the reusable medical device 190. For example, in some embodiments, the head 120 can be configured to move relative to the reusable medical device 190 when disposed within a body cavity and/or attached to a target tissue (e.g., via suction coupling). Such movement can facilitate the insertion of a distal end portion of the reusable medical device 190, the repositioning of the target tissue (e.g., the “straightening” of the cervical canal) or the like. In some embodiments, the head 120 can be configured to rotate relative to the distal end portion of the reusable medical device 190 about a single axis. In such embodiments, the coupling of the head 120 to the reusable medical device 190 can define a range of motion of the head 120 relative to the reusable medical device 190 (e.g., the reusable medical device 190 and/or the head 120 can include any number of stops, channels, guides, tabs, flanges, pivot points, etc. configured to control, direct, or otherwise influence movement of the head 120 relative to a proximal end portion (or handle portion) of the reusable medical device 190).

The head 120 includes a contact portion 122 configured to contact a surface associated with a target location. In some embodiments, the contact portion is configured to circumscribe an opening to a bodily cavity associated with the target location. For example, in some embodiments, the contact portion 122 is configured to contact a portion of a uterus to stabilize and/or maintain the uterus in a desired position during a procedure.

The head 120 defines a volume (not shown) in fluid communication with a vacuum chamber (not shown) of the reusable medical device through the vacuum line 142 and reservoir assembly 140. In this manner, a vacuum can be produced within the head 120 to secure bodily tissue to the head 120, draw a portion of a target tissue into a volume defined by and disposed within the head 120 and/or otherwise facilitate attachment of the device 100 (e.g., the contact portion 122) to the target tissue.

The reservoir assembly 140 includes a reservoir (or container or collection chamber) 143, a filter 144, and a connection member 145. The filter 144 limits bodily fluid (e.g., liquid and/or particulates) from entering the reusable medical device 190 (i.e., the handle and/or the vacuum chamber) while allowing air to pass therethrough. The filter 144 can allow air to pass through in either direction but does not allow bodily fluid or any other fluid to pass therethrough in either direction. Thus, the filter 144 allows for a vacuum to be produced within the reservoir 143 and/or the head 120 without letting bodily fluids contaminate the reusable part of the device. As described in more detail below, this arrangement facilitates the use of the disposable assembly 180 and the reusable portion 190 to maintain sterility and improve efficiency.

The filter 144 can be constructed from any suitable material. Such materials can include for example, porous or semi-porous materials configured to prevent particles above a certain size threshold from passing therethrough. For example, the filter can be constructed from an acrylic copolymer membrane. In some embodiments, the filter can have a pore size from about 0.3 micron to about 0.6 microns. In some embodiments, the filter can have a pore size of about 0.45 microns. Thus, certain biologics, cells and/or other bodily tissue can be prevented from passing into the reusable medical device 190 during operation. In other embodiments, the filter 144 can be a check valve or any other one-way valve mechanism. In this manner, the reservoir 143 can collect bodily fluids which have not passed through the filter 144. The reservoir 143 and the filter 144 can be used together or independently to retain and/or stop bodily fluids from entering the reusable medical device.

In some embodiments, the filter 144 can be disposed in a position such that a gas (e.g., air) flow path is maintained even as bodily fluid (e.g., liquid) collects in the collection chamber. The reservoir 143 can be any size suitable to collect such bodily fluids while allowing the vacuum produced in the vacuum chamber of the reusable medical device 190 to be conveyed to the head 120 of the disposable assembly 180. For example, in some embodiments, the reservoir has a volume of 2 ml. In other embodiments, the reservoir has a volume of from about 1 ml to about 3 ml, or about 3 ml to 5 ml. In other embodiments, for example when performing a procedure at a portion of a body having substantial bodily fluid, the reservoir can have a volume larger than about5 ml.

The connection member 145 is attached to the reservoir assembly 140, and can include, for example, a connection port and/or a clip. In such instances, the connection port and/or clip can be removably coupled to the distal portion of the reusable medical device 190 to create a snap fit or O-ring connection (herein referred to as “the connection”). In this manner, the reusable medical device 190 and disposable assembly 180 can be easily coupled or decoupled by push of a button or lever or any other motion of associated parts.

The reusable medical device 190 can be sealed such that water and/or disinfecting fluid cannot enter the internal components during washing or disinfecting procedure. Thus, the removable connections described herein allow the disposable and the reusable parts of a medical device to be used in concert.

The vacuum source can be any suitable device, mechanism, assembly, etc. configured to produce a negative pressure differential once actuated. For example, in some embodiments, the vacuum source can be a syringe mechanism disposed within a handle of the shaft of the type shown and described in the '335 application. In such embodiments, actuation of the vacuum source can increase a volume within the syringe, which in turn, reduces a pressure therein. Thus, the actuation of the vacuum mechanism produces a negative pressure differential between the head 120 and the vacuum mechanism that can result in a suction force being exerted within the head 120. The vacuum mechanism can for example, be substantially similar to or the same as any of the mechanisms, and/or assemblies described in the '872 publication incorporated by reference above. As such, the vacuum mechanism is not described in further detail herein. Although described as being a mechanical (or non-electronic) mechanism, in other embodiments, the vacuum mechanism can include an electric pump that produces the negative pressure.

The suction force exerted within the head 120 (e.g., during a medical procedure) is illustrated by vacuum flow path AA in FIG. 2. As shown, the vacuum flow path AA communicates from the head 120 towards the reusable medical device. Specifically, the flow path AA communicates from the vacuum source (e.g., from the vacuum chamber of the reusable medical device 190), through the reservoir assembly 140 (i.e., through the reservoir 143 and the filter 144), through the vacuum line 142 and into the volume defined by the head 120. Further, the management of bodily fluid at the reservoir assembly 140 is illustrated by bodily fluid path BB in FIG. 2. The bodily fluid path BB illustrates the transfer of bodily fluid received at the head 120 and conveyed to the reservoir 143 of the reservoir assembly 140. As shown, the bodily fluid path BB terminates at the filter 144, as described above, such that bodily fluid is limited or prevented from entering or otherwise contaminating the reusable medical device 190.

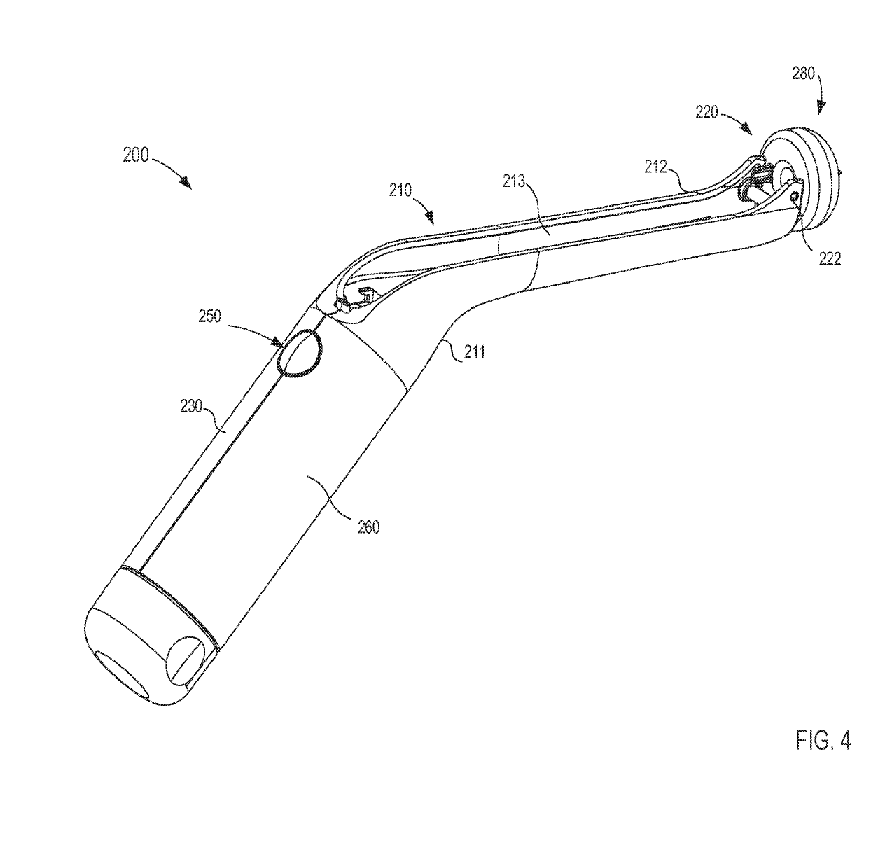

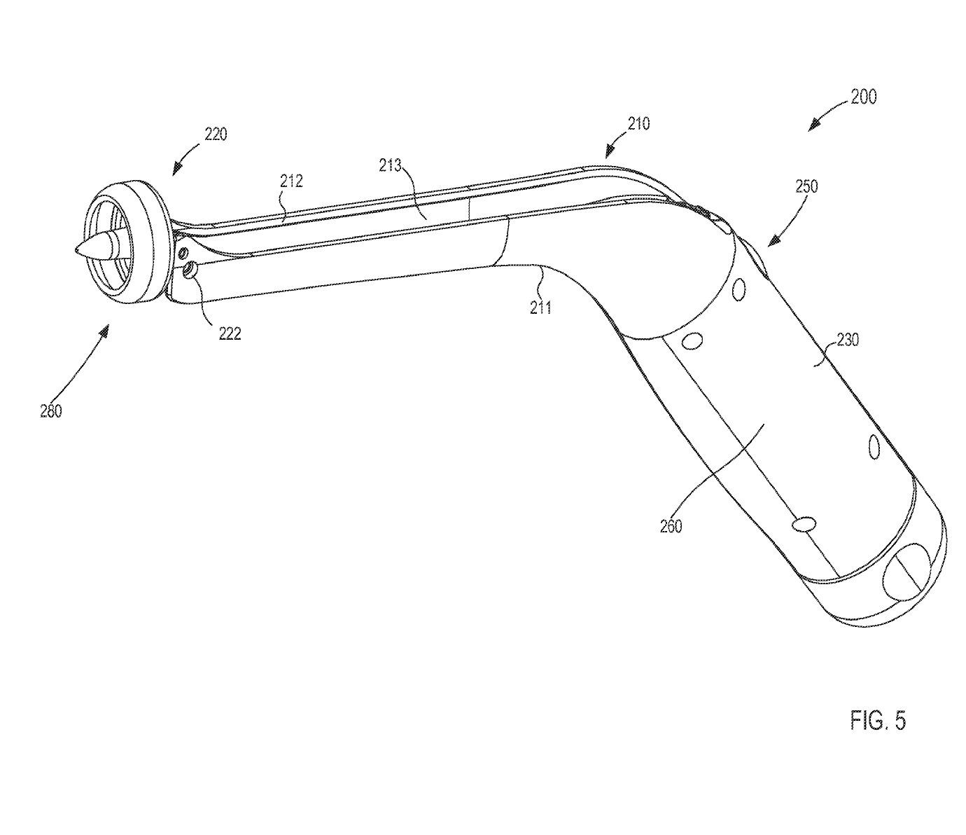

FIGS. 4-9 illustrate a medical device 200 (and portions thereof) according to an embodiment. The medical device 200 (and any of the other insertion devices and/or tissue engagement devices described herein) can be used with any of the sheaths, insertion members or the like described herein. In some embodiments, the medical device 200 can be used to engage, manipulate and/or secure a bodily tissue to facilitate a procedure on the bodily tissue. The medical device 200 (also referred to herein as “device”) includes a retractor 210 and a head assembly 280 that includes a vacuum nozzle 220 (also referred to herein as “head” or “nozzle” or “vacuum head” or “vacuum port”). The head assembly 280 can be a removable head assembly of the types shown and described herein. The vacuum nozzle 220 can be coupled to the retractor 210 for pivotal movement, as described in further detail herein. As shown in FIGS. 4 and 5, the retractor 210 includes a proximal end portion 211 and a distal end portion 212. The proximal end portion 211 can be engaged by a user to manipulate the retractor 210. The distal end portion 212 can be movably coupled to the vacuum nozzle 220, as described in further detail herein. The retractor 210 can be, for example, substantially similar to or the same as any of the retractors, body portions, housings, and/or delivery devices described in the '734 application and/or '335 application incorporated by reference above. As such, the retractor 210 is not described in further detail herein.

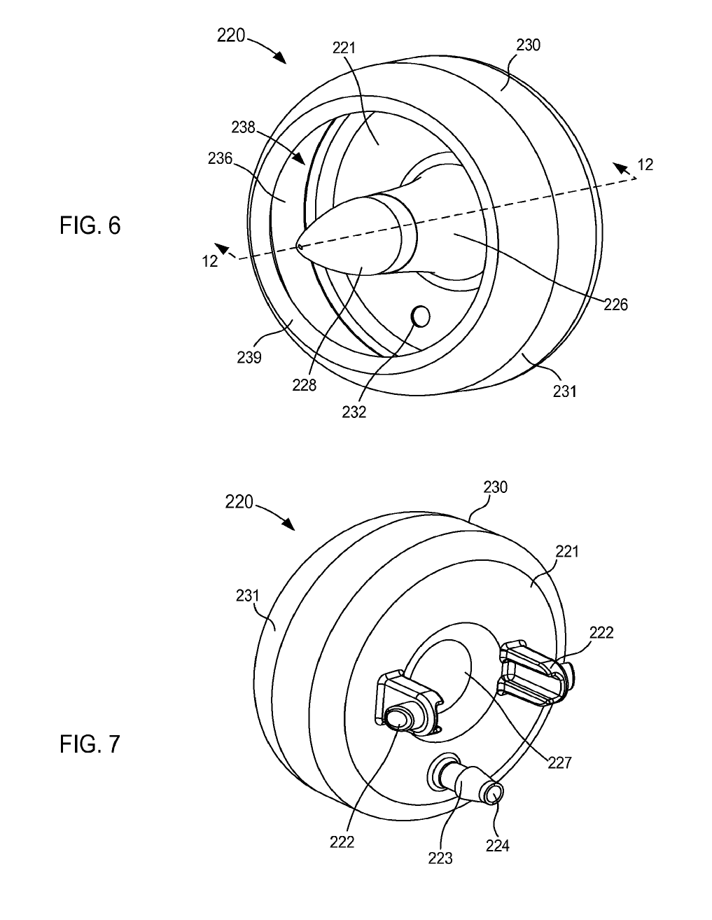

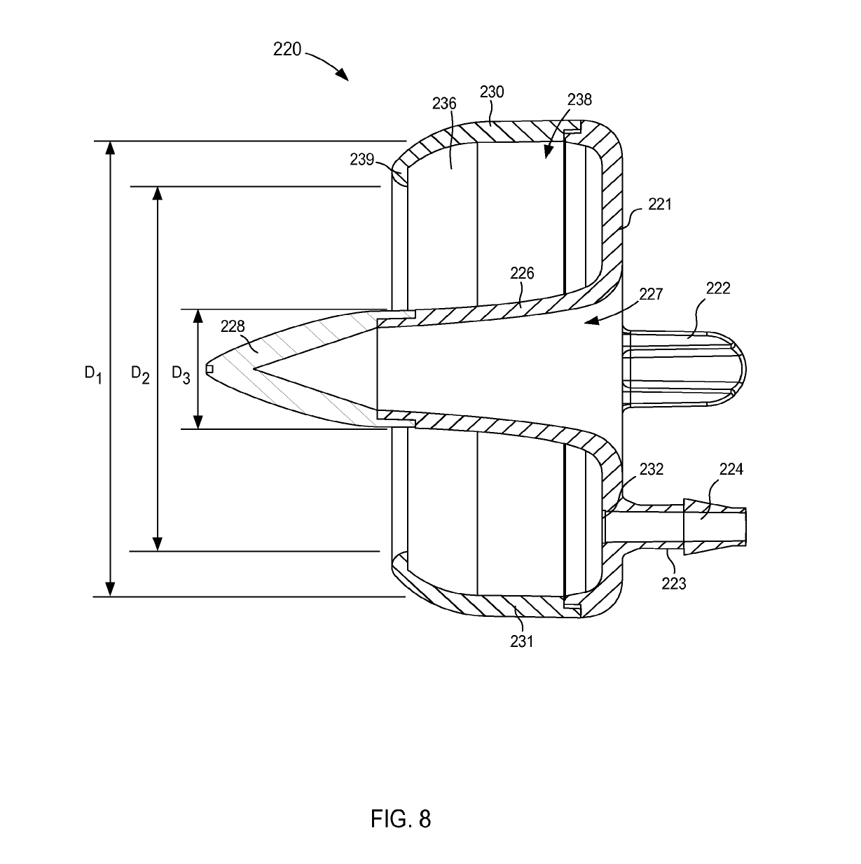

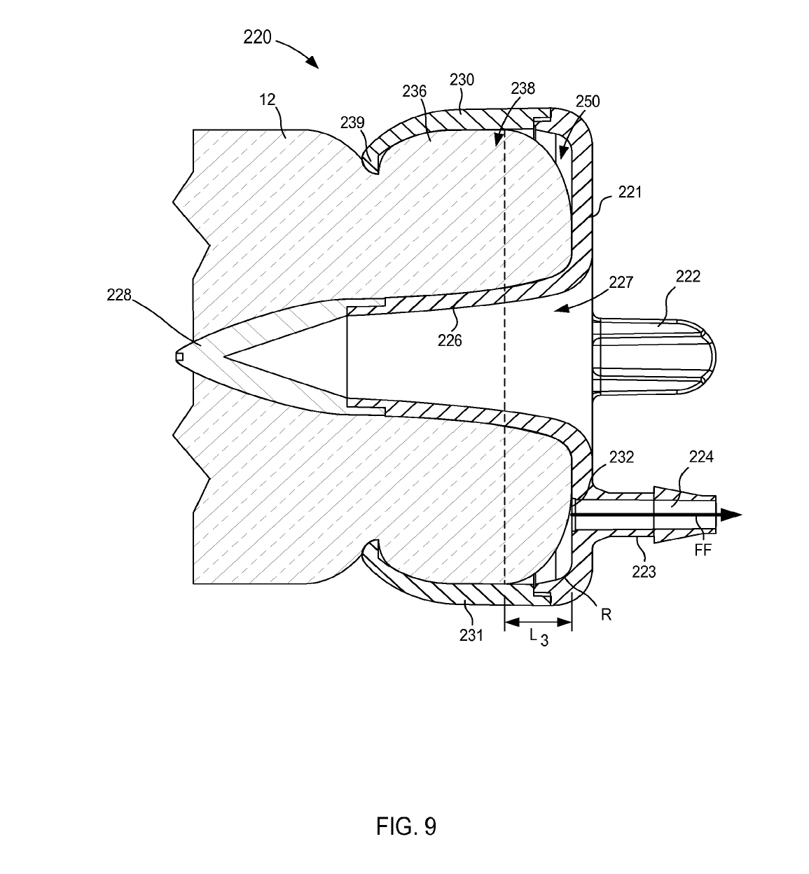

As shown in FIGS. 6-9, the vacuum nozzle 220 includes a connection portion 221 and an engagement portion 230. The connection portion 221 includes a vacuum port 223 (also referred to herein as “port”) and a set of connection members 222. The connection members 222 are configured to be movably coupled to the distal end portion 212 of the retractor 210. For example, as shown in FIG. 7, the connection members 222 can include substantially cylindrical protrusions or the like that can be disposed in a corresponding opening defined by the distal end portion 212 of the retractor to movably couple the nozzle 220 to the distal end portion 212 of the retractor 210 (see e.g., FIGS. 8 and 9). In this manner, the nozzle 220 can be configured to move relative to the retractor 210. For example, in some embodiments, the nozzle 220 can be configured to move relative to the retractor 210 when disposed within a body cavity and/or attached to a target tissue (e.g., via suction coupling). Thus, the movement can facilitate the insertion of the distal end portion 212 of the retractor 210. In some embodiments, the nozzle 220 can be configured to rotate relative to the retractor 210. In such embodiments, the coupling of the nozzle 220 to the retractor 210 can define a range of motion of the nozzle 220 relative to the retractor 210 (e.g., the retractor 210 and/or the nozzle 220 can include any number of stops, channels, guides, tabs, flanges, pivot points, etc. configured to control, direct, or otherwise influence movement of the nozzle relative to the handle 210).

The port 223 of the connection portion 221 defines a lumen 224 in fluid communication with a vacuum source (e.g., a vacuum chamber defined by a portion of the retractor 210) and a portion of the engagement portion (see e.g., FIG. 8). The vacuum source can be any suitable device, mechanism, assembly, etc. configured to produce a negative pressure differential once actuated. For example, in some embodiments, the vacuum source can be a syringe mechanism or the like disposed within a handle of the retractor 210, as described in the '335 application. In such embodiments, actuation of the vacuum source can increase a volume within a syringe or the like, which in turn, reduces a pressure therein. Thus, the actuation of the vacuum source produces a negative pressure differential between the port 223 and the vacuum source that can result in a suction force being exerted within the lumen 224, as described in further detail herein.

In some embodiments, the lumen 224 can be fluidically coupled to the vacuum source via any suitable vacuum line, filter and reservoir systems described herein. For example, in some embodiments, the head 220 and/or the lumen 224 can be fluidically coupled to a vacuum source within the handle of the retractor 210 via a removable, disposable connection, of the types shown and described herein.

As shown in FIGS. 6-9, the engagement portion 230 of the nozzle 220 is coupled to the connection portion 221 and is configured to receive a portion of a target tissue (as described in further detail herein). The engagement portion 230 can be any suitable shape, size, and/or configuration. For example, in some embodiments, the engagement portion 230 can be substantially cylindrical including and/or otherwise being formed from a set of annular walls 231. As such, the annular walls 231 include an inner surface 236 having a diameter D₁. The inner surface 236 defines a suction volume 238 configured to be in fluid communication with the suction port 223. More specifically, the engagement portion 230 defines an opening 232 that places the suction volume 238 in fluid communication with the lumen 224 of the port 223, as shown in FIG. 8. In this manner, when the vacuum source is actuated, a negative pressure (e.g., a suction force) is produced within the suction volume 238 that can be operable in retaining a target tissue within at least a portion of the suction volume 238, as described in further detail herein.

The inner surface 236 also includes and/or forms a rib 239 disposed at a distal end of the set of annular walls 231. The rib 239 can be, for example, a protrusion, a tab, a ridge, a rail, a flange, a ring, and/or like that extends from the inner surface 236 into the suction volume 238. For example, as shown in FIG. 8, the rib 239 has a diameter D₂ that is smaller than the diameter D₁ defined by the inner surface 236 (e.g., associated with the suction volume 238). As such, the rib 239 can extend from the inner surface 236 to selectively engage a portion of the target tissue when disposed in the suction volume 238 (see e.g., FIG. 9). More specifically, the diameter D₂ of the rib 239 can be such that the rib 239 deforms a portion of the target tissue when disposed in the suction volume 238, which can be operable in retaining a target tissue within the suction volume 238.

In some embodiments, the rib 239 can be substantially continuous (e.g., continuously encompasses the suction volume 238). In other embodiments, the rib 239 can include multiple portions and/or sections, defining one or more channels therebetween. The rib 239 can be any suitable shape or size. For example, in this embodiment, a distal surface of the rib 239 is substantially rounded, while a proximal surface of the rib 239 is substantially linear. Moreover, while the rib 239 is shown in FIG. 8 as extending in a substantially perpendicular direction from the inner surface 236, in some embodiments, the rib 239 can extend from the inner surface 236 at any suitable angle. Moreover, in some embodiments, the proximal surface of the rib 239 can be in-cut or the like, wherein a width of the rib 239 increases as the rib 239 extends from the inner surface 236.

The engagement portion 230 also includes an elongate portion 226 that extends from a proximal end portion of the engagement portion 230. As shown, the elongate portion 226 extends substantially though a center of the engagement portion 230. In other embodiments, an elongate portion can be offset from a center of an engagement portion. The elongate portion 226 can be any suitable shape, size, or configuration. For example, as shown in FIGS. 6, 8, and 9, the elongate portion 226 can be substantially tapered (e.g., tapered to a rounded distal end). Said a different way, the elongate portion 226 can have a diameter D₃ that decreases as the elongate portion 226 extends in the distal direction. In other embodiments, the elongate portion 226 need not be tapered. At least a portion of the elongate portion 226 can be monolithically formed with the vacuum nozzle 220. For example, the vacuum nozzle 220 can be a single molded piece. As such, the elongate portion 226 can have a stiffness that is sufficiently large to allow for insertion into a body cavity without undue deformation. For example, the elongate portion 226 can be sufficiently stiff as to resist and/or withstand an axial force exerted thereon when the elongate portion 226 enters the cervix os or other body cavity.

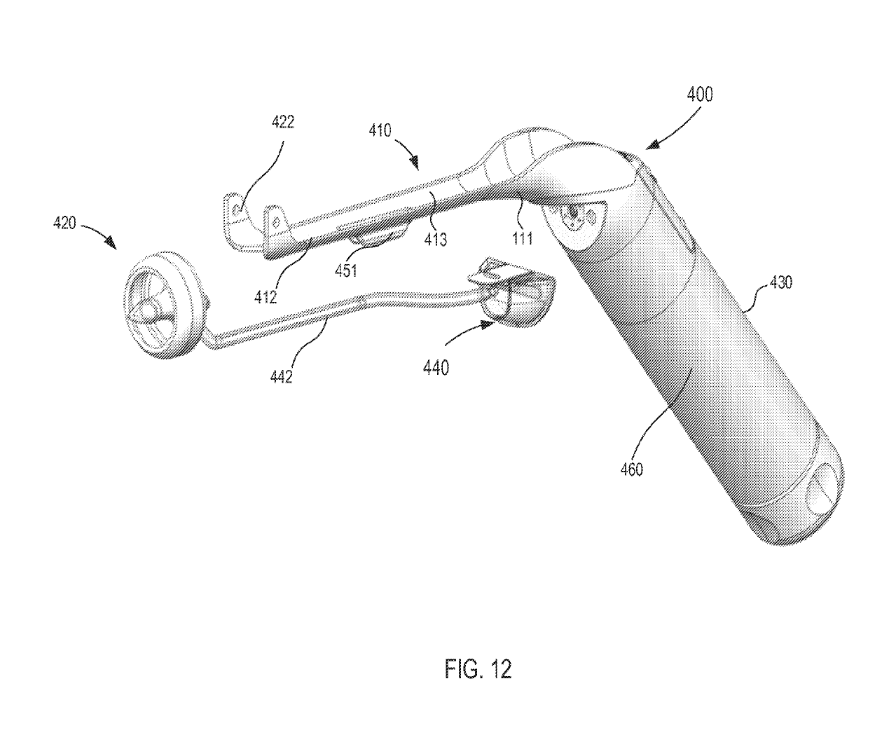

The elongate portion 226 defines a lumen 227 that is configured to receive, for example, a sheath (e.g., the sheath 160 and/or 260), an implant, a pharmaceutical, and/or any suitable portion of an insertion mechanism such as a catheter, a tube, a rod, an instrument, and/or the like. In this manner, the elongate portion 226 can allow an implant, pharmaceutical, etc. to be advanced through the suction volume 238 to be delivered to a desired portion of the body that can be, for example, in a distal position relative to the nozzle 220. Moreover, the elongate member 226 includes a distal tip 228 (or dilation member) that is at least partially disposed is a distal position relative to the rib 239 (see e.g., FIG. 8). In some embodiments, the distal tip 228 can be formed independently from the elongate portion 226 and coupled thereto. For example, the distal tip 228 can be formed from a material (e.g., silicone, siliconized rubber, rubber, and/or the like) having a durometer that is less than a durometer associated with the material forming the elongate member 226, the rib 239 and/or any other portion of the nozzle 220. Thus, the reduced durometer can, for example, allow the distal tip 228 to bend, flex, and/or otherwise deform in response to the axial force (described above), and thus, can reduce and/or substantially eliminate damage to bodily tissue during insertion of the nozzle 220. Although not shown in FIGS. 8 and 9, the distal tip 228 can be transitioned from a first configuration (e.g., a closed configuration as shown in FIG. 12) and a second configuration (e.g., an open configuration in which the lumen 227 extends therethrough). For example, in some embodiments, the distal tip 228 can include one or more dilation members or the like that can be transitioned from the first configuration, in which the dilation members are substantially closed, to the second configuration, in which the dilation members are substantially open. In some embodiments, the distal tip 228 can define one or more slits, cuts, openings, notches, and/or the like that can, for example, form at least a portion of the dilation members. Thus, a sheath, implant, pharmaceutical, and/or any other suitable portion of the insertion mechanism or the like can be passed through the lumen 227 to be delivered to a desired bodily tissue (e.g., the fundus 14 of the uterus 11 (FIG. 1)), as described above.

In use, at least a portion of the device 200 can be inserted into a body cavity and manipulated to place the nozzle 220 in contact with a target tissue. For example, in some instances, the distal end portion 212 of the retractor 210 can be inserted into the vagina 15 of a patient and advanced to place the nozzle 220 in contact with a portion of the cervix 12 (i.e., the target tissue). Once in contact with the cervix 12 (as shown in FIG. 9), the vacuum source can be actuated and in turn, a suction force can be produced in the lumen 224 of the port 223 and at least a portion of the suction volume 238 of the engagement portion 230, as indicated by the arrow FF in FIG. 9. Thus, the suction force produced within the suction volume 238 can draw a portion of the cervix 12 into the suction volume 238. Moreover, with the portion of the cervix 12 drawn into the suction volume 238, the elongate portion 226 can extend through the cervical os (not shown in FIG. 9) such that at least a portion of the distal tip 228 is positioned within the uterus (not shown in FIG. 9).

In some embodiments, the portion of the cervix 12 can be selectively placed in contact with the inner surface 236 of the engagement portion 230 when drawn into the suction volume 238. For example, as shown in FIG. 9, the cervix 12 can be drawn into the suction volume 238 and selectively placed into contact with the inner surface 236 to define a vacuum pathway 250 between a first portion of the cervix 12 and a portion of the inner surface 236. More specifically, in some embodiments, the inner surface 236 can include a proximal portion (e.g., associated with a proximal wall or the like) and a circumferential portion (e.g., associated with the set of annular walls, described above) forming an intersection portion therebetween. In some embodiments, the intersection portion formed between the proximal portion and the circumferential portion can define a curved shape having a predetermined radius of curvature R, as shown in FIG. 9. The radius of curvature R defined by the intersection portion can, for example, be sufficiently small such when the cervix 12 is drawn into the suction volume 238 and selectively placed in contact with the inner surface 236, the intersection portion is spaced apart a distance L₃ from a surface of the cervix 12, thereby defining the vacuum pathway 250. In some embodiments, the vacuum pathway 250 can be a substantially continuous volume that circumscribes a portion of the cervix 12 and that can maintain substantially continuous communication with the port 223. As such, the suction force produced by the vacuum source can be substantially consistent within the vacuum pathway 250 and can, for example, be distributed with substantial uniformity about the portion to the cervix 12. Moreover, while the cervix 12 is shown in FIG. 9 as being in contact with the elongate portion 226 at or adjacent to the proximal portion of the inner surface 226, in other embodiments, the arrangement of the elongate portion 226 can be such that the cervix 12 is similarly spaced apart from the a portion of the elongate portion 226.

As shown in FIG. 9, the cervix 12 can be drawn into the suction volume 238 such that a surface of the rib 239 is placed in contact with a surface of the cervix 12. More specifically, since the rib 239 extends from the inner surface 236 (as described above), the rib 239 can define a diameter that is smaller than a diameter of the remaining portions of inner surface 236. Thus, with the cervix 12 disposed in the suction volume 238 and selectively in contact with the inner surface 236, the rib 239 can deform a corresponding portion of the cervix 12 and as such, can place at least a portion of the proximal surface of the rib in contact with the cervix 12. Accordingly, the contact between the proximal surface of the rib 239 and the cervix 12 can limit movement of the cervix 12 in a direction away from the connection portion 221 (i.e., the distal direction). In this manner, the suction force exerted on a first portion of the cervix 12 via the vacuum pathway 250 and the contact between at least the proximal surface of the rib 239 and a second portion of the cervix 12 can retain the cervix 12 within the engagement portion 230 (e.g., the suction volume 238). Moreover, the cervix 12 can be retained within the engagement portion with a desired force sufficient to substantially prevent the cervix 12 from being withdrawn from the engagement portion 230 when the device 200 is manipulated to exert a traction force on the cervix 12. As such, the cervix 12 can be manipulated, moved, and/or otherwise reoriented to facilitate the insertion of, for example, an implant or the like. For example, in some instances, the nozzle 220 can retain a portion of the cervix 12 in the engagement portion 230 (e.g., the suction volume 238), while a traction force is applied thereto, thereby facilitating access (e.g., for a sheath or other delivery mechanism) through the cervical os 13 and into the uterus 11.

In some embodiments, an amount of suction force exerted on the cervix 12 can be increased or decreased by changing the arrangement of the engagement portion 230. For example, in some embodiments, the size of the rib 239 can be increased or decreased to increase or decrease, respectively, a contact surface between the portion of the cervix 12 and, for example, the proximal surface of the rib 239. For example, in some embodiments, the diameter D₂ defined by the rib 239 can be decreased. Accordingly, an increase in a size of the contact surface can, for example, result in an increase in a force configured to resist the distal movement of the cervix 12 relative to the engagement portion 230 (as described above) without a need, for example, to increase a suction force (e.g., an increase in a negative pressure differential produced by the vacuum source).

In a similar manner, an increase in a volume of the suction volume 238 and/or the vacuum pathway 250 can increase a force exerted on the cervix 12 to retain the vacuum nozzle 220 in contact with the cervix 12 at higher pull forces. Thus, by increasing the volume of the suction volume 238 and/or the vacuum pathway 250 a suction force as result of a negative pressure differential produced by the vacuum source 490 can be reduced, while still retaining the cervix 12 within the engagement portion 230 during traction. For example, in some embodiments, a cross-sectional area of the suction volume 238 can be increased or decreased to increase or decrease, respectively, a force to retain the cervix 12 in the engagement portion 230. By way of example, in some embodiments, the diameter D₃ of the elongate portion 226 can be decreased and as such, the suction volume 238 defined between the elongate portion 226 and the inner surface 236 can be increased. In other embodiments, a depth of the suction volume 238 can be increased or decreased to increase or decrease, respectively, a force to retain the cervix 12 in the engagement portion 230. In this other embodiments, the radius of curvature defined by the transition portion of the inner surface can be increased or decreased to increase or decrease, respectively, a force to retain the cervix 12 in the engagement portion 230.

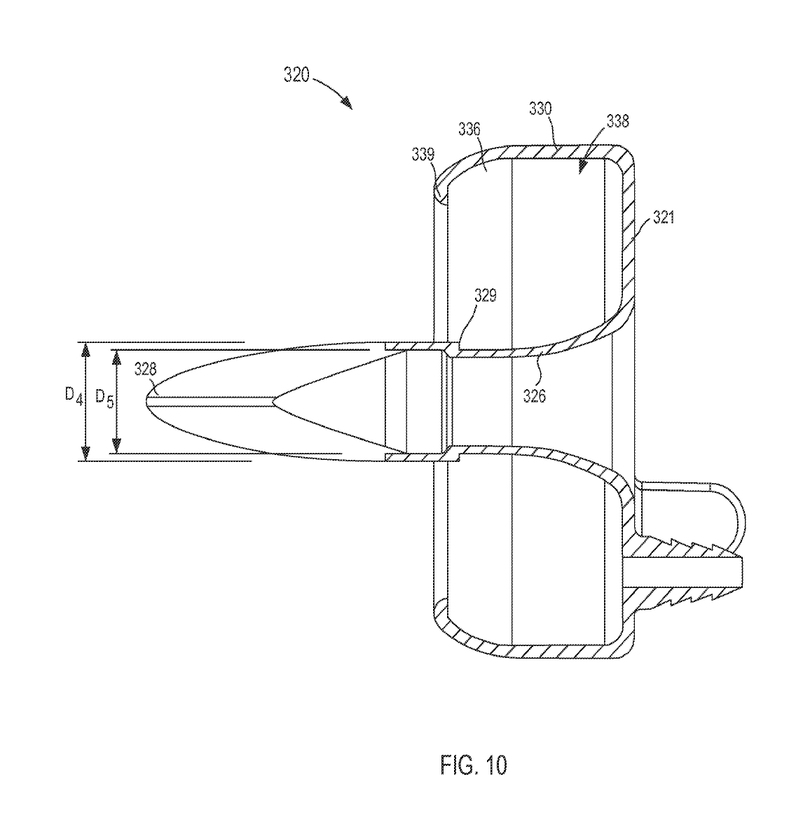

Although the vacuum nozzle 220 is shown in FIGS. 6-9 as including multiple pieces that are joined together, in other embodiments, the vacuum nozzle (or head) 220 (and any of the heads described herein) can be monolithically constructed. Although the vacuum nozzle 220 is shown in FIGS. 6-9 as including the elongate portion 226 with the diameter D₅ that forms a substantially smooth taper as the elongate portion 226 extends in the distal direction, in other embodiments, a vacuum nozzle can include an elongate portion that can form, for example, a rib or the like. By way of example, FIG. 10 is an illustration of a vacuum nozzle 320 according to another embodiment. The vacuum nozzle 320 includes a connection portion 321 and an engagement portion 330. The vacuum nozzle 320 can be substantially similar in form and/or function as the vacuum nozzle 320 described above with reference to FIGS. 4-9. Thus, aspects of the vacuum nozzle 320 that are similar to corresponding aspects of the vacuum nozzle 220 are not described in further detail herein. The vacuum nozzle 220 can differ from the vacuum nozzle 320, however, in the arrangement of the engagement portion 330. More specifically, as shown in FIG. 10, the engagement portion 330 includes an inner surface 336 that includes and/or forms a first rib 339 and that defines a suction volume 338 (e.g., similar to the rib 239 and the suction volume 238, respectively, included in the vacuum nozzle 220). The engagement portion 330 also includes an elongate portion 326 that extends from a proximal portion of the inner surface 536 and that is coupled to a distal tip 328 (e.g., similar to the distal tip 228 of the vacuum nozzle 220). As shown, the elongate portion 326 includes a second rib 329 that extends from a surface of the elongate portion 326 toward the first rib 339. In this manner, the second rib 329 can have a first diameter D₄ that is greater than a second diameter D₅ of at least a portion of the elongate portion 326 that is proximal to the second rib 329. Thus, the first rib 339 and the second rib 329 can be configured to collectively engage a portion of a target tissue when the target tissue is disposed in the suction volume 338, as described in detail above with reference to the nozzle 220.

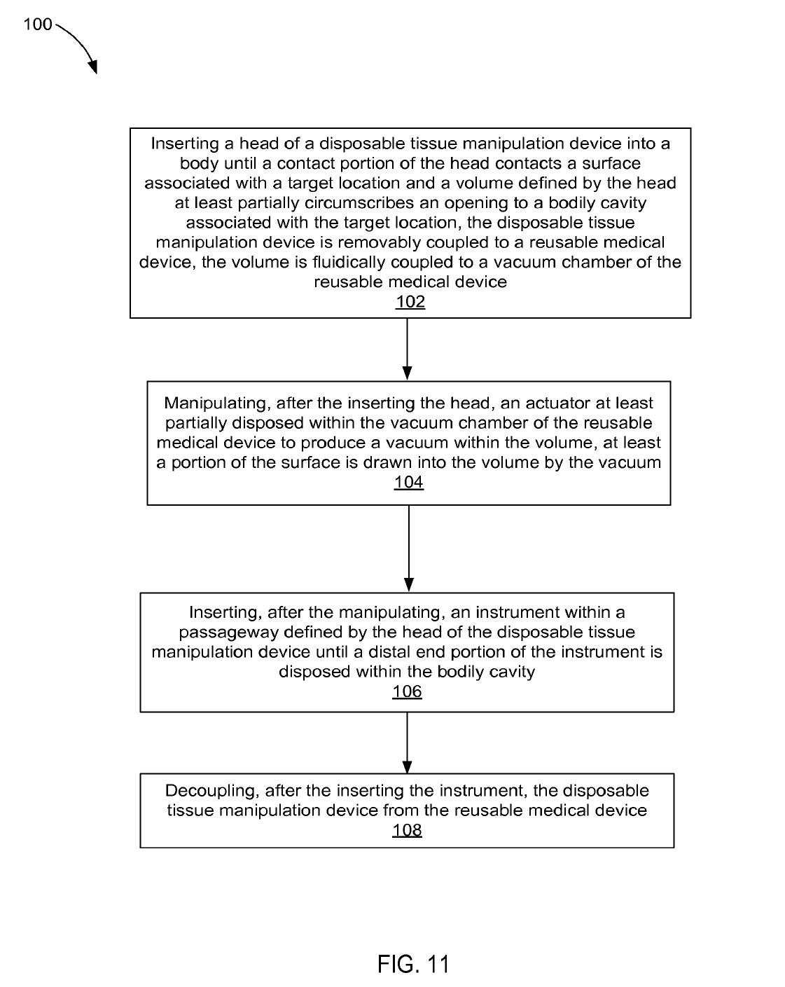

FIG. 11 shows a schematic flow diagram of a method 100 of inserting an instrument into a bodily cavity using a disposable tissue manipulation device and a reusable medical device. The method 100 includes inserting a head of a disposable tissue manipulation device (e.g., the disposable assembly 180 or any other disposable component described herein) into a body until a contact portion of the head (the head 120 or any other head described herein) contacts a surface associated with a target location, at 102. The disposable tissue manipulation device is removably coupled to a reusable medical device (e.g., the reusable medical device 190 or any other reusable medical device described herein). The volume is fluidically coupled to a vacuum chamber of the reusable medical device.

The method 100 further includes manipulating, after the inserting the head, an actuator at least partially disposed within the vacuum chamber of the reusable medical device to produce a vacuum within the volume, at 104. At least a portion of the surface is drawn into the volume by the vacuum. In this manner, as described herein, the bodily tissue can be engaged, manipulated and/or secured to facilitate a procedure associated with the bodily tissue.

The method 100 further includes inserting, after the manipulating, an instrument within a passageway defined by the head of the disposable tissue manipulation device until a distal end portion of the instrument is disposed within the bodily cavity, at 106. After inserting the instrument, the method 100 further includes decoupling the disposable tissue manipulation device from the reusable medical device, at 108. In this manner, the disposable tissue manipulation device, including any contamination disposed thereon and/or collected therein (e.g., via the reservoir assembly) from the medical procedure, can be properly and safely disposed of. Further, the reusable medical device can be suitably cleaned and prepared for a subsequent procedure using another disposable tissue manipulation device. Similarly stated, the reusable medical device can be repeatedly removably coupled to an unused or sterile tissue manipulation device when the reusable medical device is used for multiple procedures.

In some embodiments, the method 100 can further include inserting the instrument after verification that the vacuum is above a predetermined threshold. The verification can be based on a vacuum gauge of the reusable medical device. In this manner, a medical practitioner can suitably determine when insert the instrument via the reusable medical device.

FIGS. 12-17 illustrate a medical device 400 according to an embodiment. The medical device 400 (and any of the other insertion devices and/or tissue engagement devices described herein) can be used with any of the sheaths, insertion members or the like described herein or in the '872 publication, the '452 PCT publication and/or the '351 PCT publication, which are incorporated by reference above. in some embodiments, the medical device 400 can be used to engage, manipulate and/or secure a bodily tissue to facilitate a procedure on the bodily tissue. The medical device 400 (also referred to herein as “device”) includes a shaft 410, a vacuum port (also referred to herein as “head”) 420, a handle 430, a reservoir assembly 440, a connection member 445, and vacuum mechanism 460. In some embodiments, the device 400 can optionally include a vacuum gauge 450. As shown in FIGS. 12-15, the shaft 410 includes a proximal end portion 411, a distal end portion 412 and a delivery passageway 413. The proximal end portion 411 can be engaged by a user (via the handle 430) to manipulate the shaft 410. The distal end portion 412 can be movably coupled to the vacuum port 420. The delivery passageway 413 provides a passageway through which any suitable tools (e.g., inserters or the like) can be disposed to access the target bodily tissue. The shaft 410 can be, for example, substantially similar to or the same as any of the retractors, body portions, housings, and/or delivery devices described in the '872 publication, the '452 PCT publication and/or the '351 PCT publication incorporated by reference above. As such, the shaft 410 is not described in further detail herein.

The vacuum port 420 can be removably coupled to the shaft 410 for pivotal movement. In particular, the vacuum port 420 includes a set of connection members (or protrusions) that are received within corresponding openings 422 of the shaft 410. The connection members can include substantially cylindrical protrusions or the like that can be disposed in the corresponding openings 422 defined by the distal end portion 412 of the shaft to movably couple the port 420 to the distal end portion 412 of the shaft 410. For example, in some embodiments, the port 420 can be configured to move relative to the shaft 410 when disposed within a body cavity and/or attached to a target tissue (e.g., via suction coupling). Such movement can facilitate the insertion of the distal end portion 412 of the shaft 410, the repositioning of the target tissue (i.e., the “straightening” of the cervical canal) or the like. In some embodiments, the port 420 can be configured to rotate relative to the shaft 410 about a single axis. In such embodiments, the coupling of the port 420 to the shaft 410 can define a range of motion of the port 420 relative to the shaft 410 (e.g., the shaft 410 and/or the port 420 can include any number of stops, channels, guides, tabs, flanges, pivot points, etc. configured to control, direct, or otherwise influence movement of the port relative to the handle 410).

The vacuum port 420 defines a lumen (not shown) in fluid communication a vacuum mechanism 460 (shown as being within the handle 430) through a vacuum line 442 and reservoir assembly 440. In this manner, a vacuum can be produced within the vacuum port to secure bodily tissue to the vacuum port 420, draw a portion of a target tissue into the vacuum port 420 and/or otherwise facilitate attachment of the device 400 to the target tissue. The vacuum line 442 can be removably coupled to the shaft 410 via the vacuum line attachment member 451. The vacuum line attachment member 451 is connected to shaft 410 and provides an interference or “snap” fit to retain the vacuum line 442 during use.

The vacuum source 460 can be any suitable device, mechanism, assembly, etc. configured to produce a negative pressure differential once actuated. For example, in some embodiments, the vacuum source can be a syringe mechanism disposed within the handle 430 of the shaft 410 of the type shown and described in the '335 application. In such embodiments, actuation of the vacuum source can increase a volume within the syringe, which in turn, reduces a pressure therein. Thus, the actuation of the vacuum mechanism 460 produces a negative pressure differential between the vacuum port 420 and the vacuum mechanism 460 that can result in a suction force being exerted within the vacuum port 420. The vacuum mechanism 460 can for example, be substantially similar to or the same as any of the mechanisms, and/or assemblies described in the '872 publication incorporated by reference above. As such, the vacuum mechanism 460 is not described in further detail herein. Although described as being a mechanical (or non-electronic) mechanism, in other embodiments, the vacuum mechanism 460 can include an electric pump that produces the negative pressure.

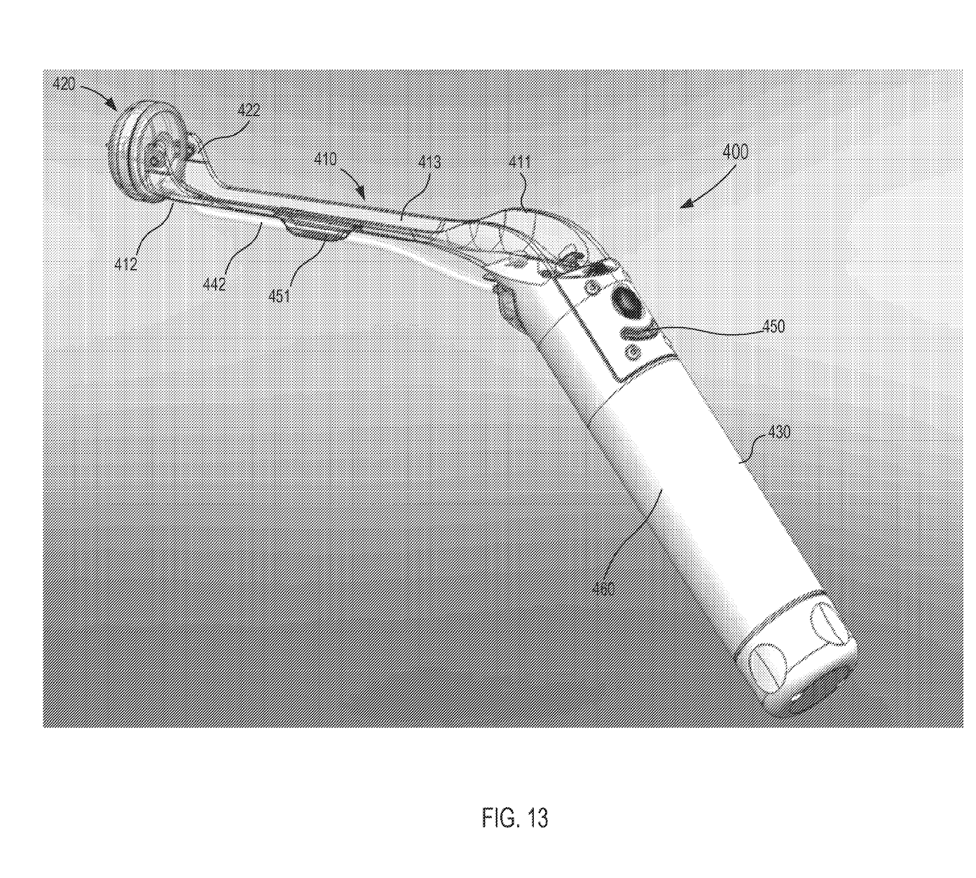

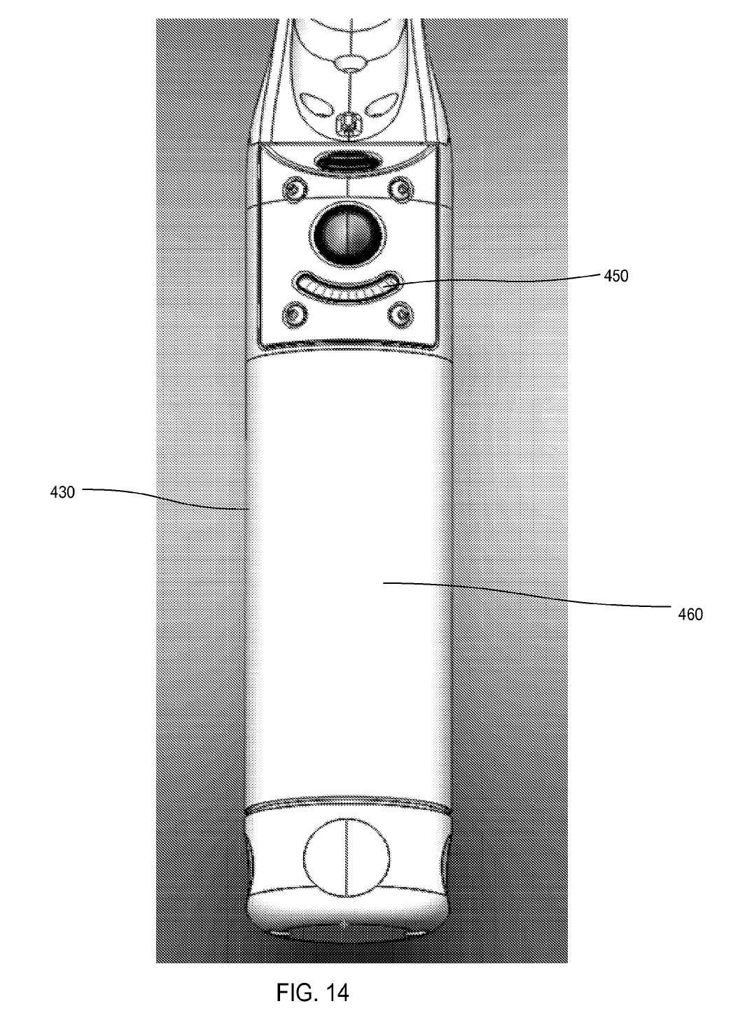

In some embodiments, the device 400 can include a vacuum gauge 450 (as shown in FIGS. 13 and 14). The vacuum gauge 450 can function as an indicator to show that the desired amount of suction is being created at the vacuum port 420 or any other part that engages the cervix or any other bodily tissue. The vacuum gauge 450 can also facilitate indication of the appropriate vacuum created by the device to the user such that the user knows when to stop actuating the vacuum source 460. The vacuum gauge 450 can work in a parallel or a serial configuration with a suction mechanism while a user activates the suction mechanism to create vacuum.

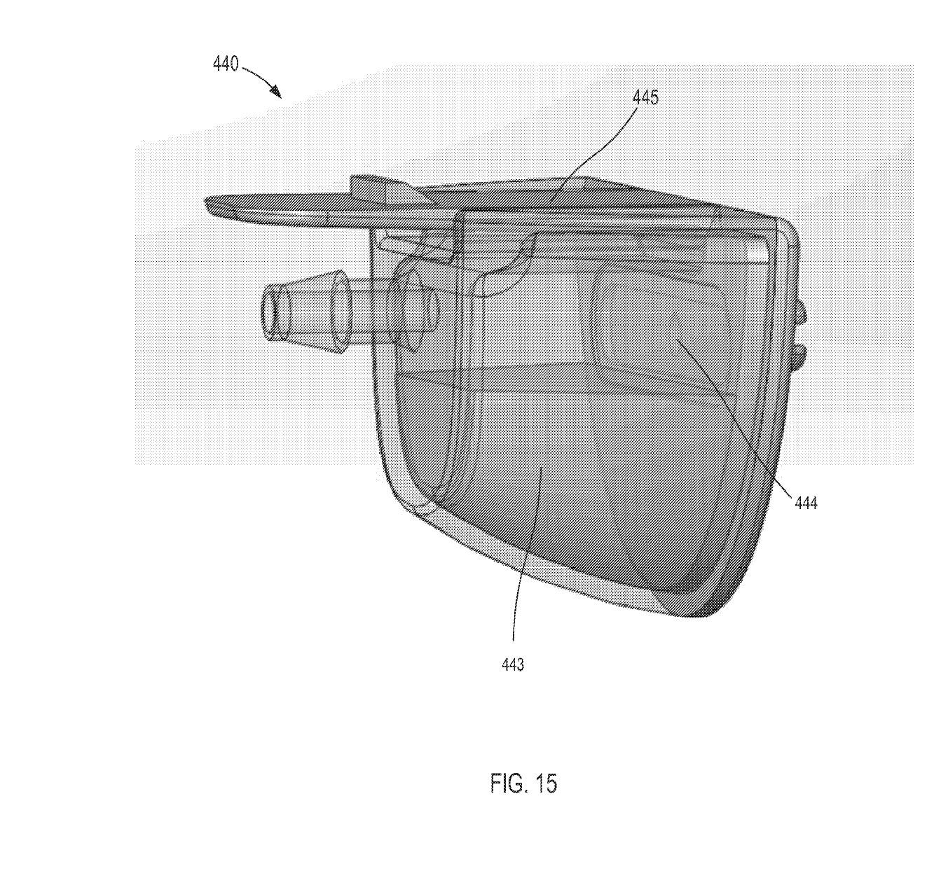

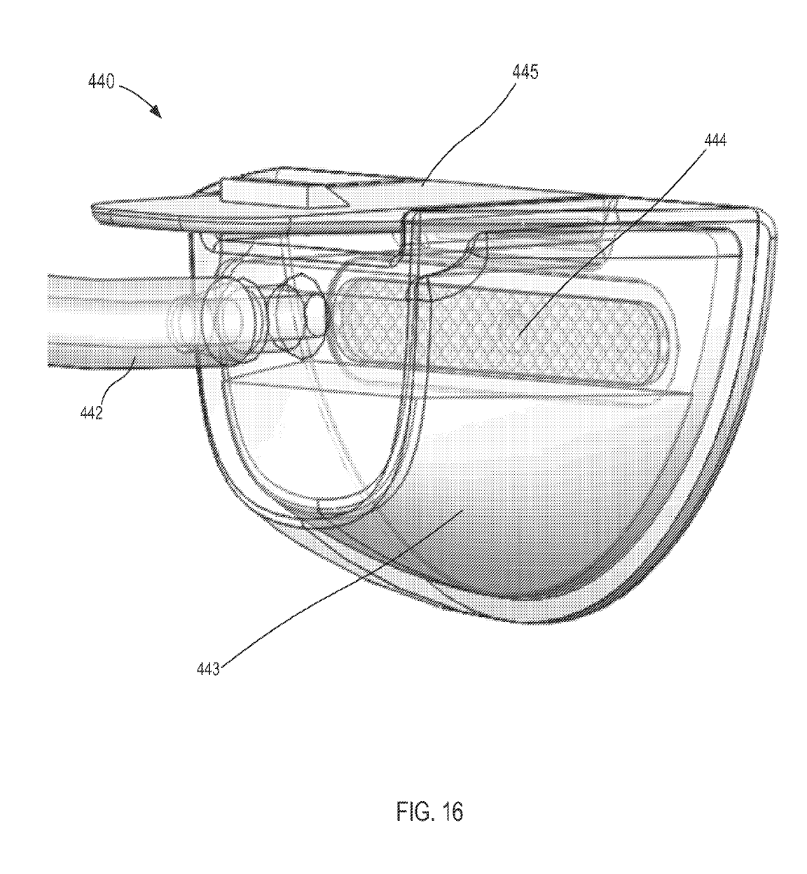

The reservoir assembly 440 (as shown in FIGS. 15 and 16) includes a reservoir (or container) 443, a filter 444, and a connection member 445. The filter 444 stops bodily fluid from entering the reusable portion of the medical device 400 (i.e., the handle 430 and/or the vacuum source 460) while allowing air to pass through. The filter 444 can allow air to pass through in either direction but does not allow bodily fluid or any other fluid to pass therethrough in either direction. Thus, the filter 444 allows for a vacuum to be produced within the reservoir 443 and/or the vacuum port 420 without letting bodily fluids contaminate the reusable part of the device. The filter 444 can by any suitable device constructed from any suitable material. Such materials can include for example, porous or semi-porous materials configured to prevent particles above a certain size threshold from passing therethrough. Thus, certain biologics, cells and/or other bodily tissue can be prevented from passing into the handle 430 and/or vacuum source 460 during operation. In other embodiments, the filter 444 can be a check valve or any other one-way valve mechanism. In this manner, the reservoir 443 can collect bodily fluids which have not passed through the filter 444. The reservoir 443 and the filter 444 can be used together or independently to stop bodily fluids from entering the reusable part of the device.

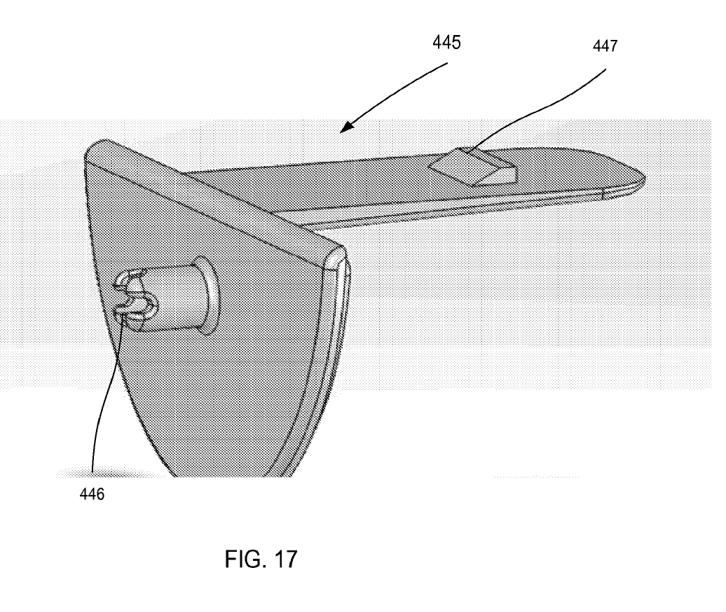

As shown in FIG. 17, the connection member 445 is attached to the reservoir assembly 440, and includes a connection port 446 and a clip 447. The connection port 446 and clip 447 can be removably coupled to the distal portion 411 of the shaft 410 to create a snap fit or O-ring connection (herein referred to as “the connection”). In this manner, the reusable and disposable parts can be easily coupled or decoupled by push of a button or lever or any other motion of associated parts. In some embodiments, the disposable parts of medical device 400 can be any of the following: the vacuum port 420, the vacuum line 442, and/or the reservoir assembly 440.

The reusable parts of the device can be sealed such that water and/or disinfecting fluid cannot enter the internal components during washing or disinfecting procedure. Thus, the removable connections described herein allow disposable and reusable parts of a medical device to be used in concert.

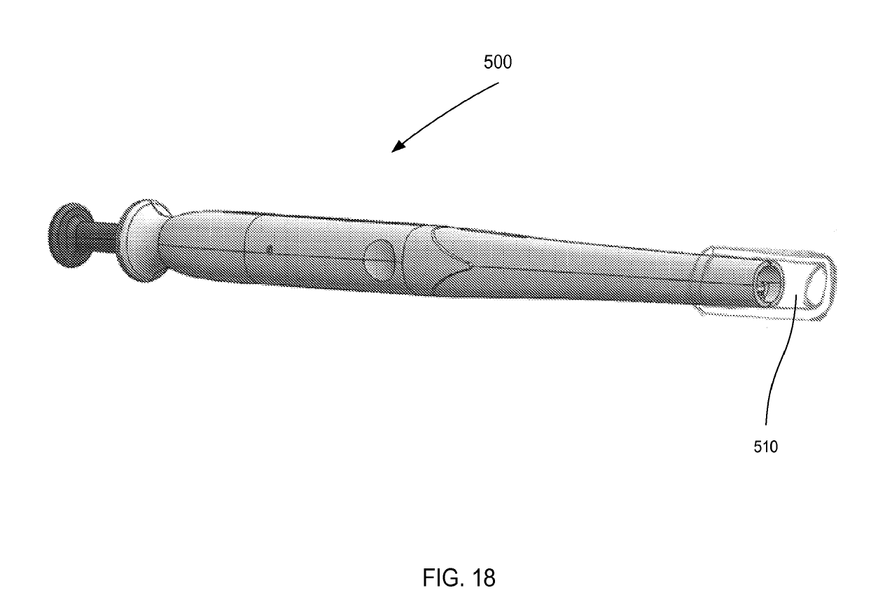

FIG. 18 illustrates an instrument 500 including a “resetting” plug 510 according to an embodiment. The instrument 500 can be any inserter, probe or the like described herein or in the '872 publication, the '452 PCT publication, and/or the '351 PCT publication. For example, in some embodiments, the instrument 500 can be an IUD inserter that is used in conjunction with the device 400, or any device described herein. After use, the instrument 500 or portions thereof can be sterilized and/or reused. The plug 510 can reset and/or seal the reusable part of the instrument 500 to allow for disinfection or washing such that no liquid can enter the device. The plug 510 is a separate from the instrument 500, but can be attached to resetting instrument 500 (shown in FIG. 18). In some embodiments, the plug 510 can include one or more protrusions configured to reset a spring, a sheath, a cutting mechanism or the like within the instrument 500 when the plug is placed thereon.

The embodiments and/or components described herein can be packaged independently or any portion of the embodiments can be packaged together as a kit. For example, in some embodiments, a kit can include a reusable medical device (e.g., such as the reusable medical device 190 described above with respect to FIG. 2, or any other reusable medical device described herein), and any number of suitable disposable assemblies (e.g., such as the disposable assembly 180 described above with respect to FIG. 2, or any other disposable assembly described herein). The reusable medical device included in the kit can define a vacuum chamber and an actuator at least partially disposed within the vacuum chamber such that movement of the actuator within the vacuum chamber produces a vacuum.

The disposable assemblies included in the kit can be configured to be removably coupled to the reusable medical device. Each disposable assembly includes a head (e.g., such as the head 120 described above with respect to FIG. 2, or any other head described herein) and a reservoir assembly (e.g., such as the reservoir assembly 140 described above with respect to FIG. 2, or any other reservoir assembly described herein). In some embodiments, the disposable assemblies included in the kit can each have a size different from the size of the other disposable assemblies included in the kit. For example, in some embodiments, a head of a first disposable assembly included in the kit can have a first nominal diameter, and a head of a second disposable assembly included in the kit can have a second nominal diameter different from the first nominal diameter. In this manner, a suitable disposable assembly having a suitable head can be selected, for example, based on the particular anatomy of a patient. In a similar manner, each reservoir assembly of the disposable assemblies included in the kit can have a size (e.g., volume; filter specifications) different from the size of the other reservoir assemblies included in the kit. In this manner, a suitable disposable assembly have a suitable reservoir assembly can be selected by a user, for example, based on the particular anatomy of a patient.

Each head (or head assembly) included in the kit can be configured to be fluidically coupled to the vacuum chamber. Further, each head defines a volume configured to at least partially circumscribe an opening to a bodily cavity associated with a target location. As described above with respect to various sized heads, the volume of each head included in the kit can be different from the volume of the other heads included in the kit. Further, each head can be configured to exert the vacuum produces by the vacuum chamber on a surface of the target location via the volume.

Each reservoir assembly is configured to be coupled to a head included in the kit, and is configured to be in fluid communication with the vacuum chamber of the reusable medical device when the reservoir assembly is coupled to the reusable medical device. Expanding further, each reservoir assembly includes a filter configured to (1) limit passage therethough of particles having a size above a threshold value (e.g., about 0.45 micron) and (2) allow air to pass therethough sufficient to maintain the vacuum within the disposable assembly.

In some embodiments, the kit can further include a sterile packaging defining a sterile volume. In such embodiments, each disposable assembly can be disposed within the sterile volume of the sterile packaging. In some instances, each disposable assembly can be disposed within its individual sterile packaging, while in other instances, multiple disposable assemblies can be disposed within a single sterile packaging. In either instance, the reusable medical device can be packaged in the kit separately from each disposable assembly (e.g., outside of the sterile packaging).

In some embodiments, as described above with respect to the sterile packaging, the kit can include the reusable medical device packaged separately from one or more of the disposable assemblies, in other embodiments, the kit can include the reusable medical device coupled to a disposable assembly. Similarly, the components of each disposable assembly (e.g., the head; the reservoir assembly; the reservoir; the filter) can, in some embodiments, be included decoupled in the kit such that a medical practitioner can assemble the same after receiving the kit. In other embodiments, such components can be coupled or otherwise assembled in the kit such that, for example, the medical practitioner can remove the disposable assembly from the sterile packaging (when included) and coupled the assembled disposable assembly to the reusable medical device.

In some embodiments, one or more instruments (or inserters) can be included in the kit. For example, in such embodiments, the kit can include any of the instruments or inserters described herein and in the '872 publication, the '452 PCT publication, and/or the '351 PCT publication, which are incorporated by reference above.

In some embodiments, the insertion members and retractors described herein (e.g., the device 100) can be used as a separate device, apart from any other specific tools and/or instruments (e.g., instrument 200). Thus, in some embodiments, the insertion members and/or retractors described herein can function substantially independently to perform functions similar to those performed by the uterine sound, cervical dilator, and/or os finder in other intrauterine procedures, including, but not limited to, artificial insemination (intrauterine insemination), colcoscopy, dilation and curettage, manual vacuum aspiration, electric vacuum aspiration, endometrial biopsy, dilation and evacuation, insertion of various contraceptive devices, and certain abortion procedures.

The embodiments described herein can be formed or constructed from a substantially flexible material (e.g., a relatively high durometer rubber, siliconized rubber, polypropylene, polyethylene and/to the like) that can allow for bending, twisting, opening, and/or otherwise reconfiguring of the distal tip. For example, the distal tip can be sufficiently flexible to be transitioned from a substantially closed configuration to a substantially opened configuration when a device, pharmaceutical, tube, rod, instrument, etc. is passed there through.

While various embodiments have been described above, it should be understood that they have been presented in a way of example only, and not limitation. Where schematics and/or embodiments described above indicate certain components arranged in certain orientations or positions, the arrangement of components may be modified. While the embodiments have been particularly shown and described, it will be understood that various changes in form and details may be made. For example, varying the shape of an aperture or using combinations of the embodiments described herein can create an alternate embodiment. Such an embodiment can form an apparatus for inserting a device into a bodily passageway, such as the cervical canal, and prevent trauma to the surrounding tissue during insertion and reduce pressure on bodily tissue when detecting tissue with the distal tip of the apparatus.

For example, although the device 400 is shown and described above as including the vacuum port 420, the vacuum line 442 and the reservoir assembly 440 that are removably coupled to the shaft 410, in other embodiments at least a portion of the shaft 410 can be removably coupled to the device 400. In such embodiments, a portion of the shaft 410 (e.g., the distal end portion 412) can be removed and disposed after each use. Such embodiments can include, for example, a two-piece shaft 410.

Although the device 400 is shown and described as including a reservoir assembly 440 that is coupled to the handle 430 via a substantially fluid-tight connection port, in other embodiments, a device can include a reservoir assembly (or parts thereof) that is directly coupled to and/or integral with the vacuum port 420. For example, in some embodiments, a vacuum port can include a reservoir and filter, such that removal of the vacuum port 420 from the shaft 410 removes the reservoir and filter. In such embodiments, the vacuum port 420 can be coupled to the vacuum source 460 via a line similar to the vacuum line 442. In such embodiments, the vacuum line 442 need not be removable from the device 400 between uses.

In some embodiments, a kit can include a shaft 210, a handle 230 (including the vacuum source 260), and a series of vacuum ports and reservoir assemblies (similar to the vacuum port 420 and the reservoir assembly 440). In this manner, the kit can include one resusable component (i.e., the shaft and handle) and multiple sets of single-use (or disposable) components). Additionally, in some embodiments, the series of vacuum ports can include a vacuum port having a first size associated with a first anatomical size of a first patient and a second vacuum port having a second size associated with a second anatomical size of a second patient. In this manner, the kit can accommodate procedures on a variety of patients of different sizes.

Although various embodiments have been described as having particular features and/or combinations of components, other embodiments are possible having a combination of any features and/or components form any of the embodiments as discussed above.

What is claimed:

1. An apparatus, comprising:
a vacuum line defining a fluid passageway and having a first end portion and a second end portion; and
a head coupled to the first end portion of the vacuum line, the head configured to be removably coupled to a first portion of a reusable medical device, the head including a contact portion configured to contact a surface associated with a target location, the contact portion configured to circumscribe an opening to a bodily cavity associated with the target location, the head defining a volume through which a vacuum is conveyed to the surface associated with the target location, the volume being in fluid communication with the fluid passageway of the vacuum line.

2. The apparatus of claim 1, further comprising:
a reservoir coupled to the second end portion of the vacuum line, the reservoir in fluid communication with the fluid passageway of the vacuum line, the reservoir configured to be removably coupled to a second portion of the reusable medical device, the reservoir including a filter configured to (1) limit passage therethrough of particles having a size above a threshold value, and (2) allow air to pass therethrough, when the reservoir is coupled to the reusable medical device.

3. The apparatus of claim 1, wherein:
the target location is a uterus,
the contact portion of the head is configured to contact an external surface of the uterus.

4. The apparatus of claim 1, wherein:
the head includes a connection member configured to mate with a corresponding connection member of the reusable medical device such that the head can rotate relative to the reusable medical device with at least one degree of freedom.

5. The apparatus of claim 1, wherein:
the corresponding connection member of the reusable medical device is an opening from a plurality of openings defined by the first portion of the reusable medical device, the connection member of the head is a protrusion from a plurality of protrusions, the protrusion from the plurality of protrusions configured to be received within the opening from the plurality of openings of the reusable medical device.

6. The apparatus of claim 1, wherein:
the head is configured to receive an insertion member therethrough when the head is coupled to the first portion of the reusable medical device and the contact portion is in contact with the surface associated with the target location.

7. The apparatus of claim 2, wherein:
when the reservoir is coupled to the reusable medical device and the contact portion of the head is in contact with the surface associated with the target location, (1) the volume of the head, (2) the fluid passageway of the vacuum line, and (3) the reservoir are in fluid communication with a vacuum chamber of the reusable medical device such that when an actuator at least partially disposed within the vacuum chamber is moved relative to the vacuum chamber the vacuum is produced and exerted on the surface associated with the target location.

8. The apparatus of claim 2, wherein:
the head is configured to be removably coupled to the first portion of the reusable medical device via a first interference fit, the reservoir being configured to be removably coupled to the second portion of the reusable medical device via a second interference fit.

9. The apparatus of claim 2, wherein:
the reservoir defines a collection chamber configured to retain bodily fluid associated with the target location and received via the fluid passageway of the vacuum line during a medical procedure, the filter of the reservoir being configured to isolate the bodily fluid within the collection chamber from the reusable medical device when the vacuum is (1) produced within the resuable medical device and (2) exerted on the surface associated with the target tissue during the medical procedure.

10. The apparatus of claim 2, further comprising:
a sterile packaging defining a sterile volume,
(1) the vacuum line, (2) the head, and (3) the reservoir disposed in the sterile volume of the sterile packaging.

11. A method, comprising:
inserting a head of a disposable tissue manipulation device into a body until a contact portion of the head contacts a surface associated with a target location and a volume defined by the head at least partially circumscribes an opening to a bodily cavity associated with the target location, the disposable tissue manipulation device removably coupled to a reusable medical device, the volume fluidically coupled to a vacuum chamber of the reusable medical device;
manipulating, after the inserting the head, an actuator at least partially disposed within the vacuum chamber of the reusable medical device to produce a vacuum within the volume, at least a portion of the surface being drawn into the volume by the vacuum;
inserting, after the manipulating, an instrument within a passageway defined by the head of the disposable tissue manipulation device until a distal end portion of the instrument is disposed within the bodily cavity; and
decoupling, after the inserting the instrument, the disposable tissue manipulation device from the reusable medical device.

12. The method of claim 11, wherein the disposable tissue manipulation device is a first disposable tissue manipulation device, the method further comprising:
coupling, after the decoupling, a second disposable tissue manipulation device to the reusable medical device.

13. The method of claim 12, wherein the head of the first disposable tissue manipulation device has a first nominal diameter, a head of the second disposable tissue manipulation device having a second nominal diameter different from the first nominal diameter.

14. The method of claim 11, wherein:
the target location is a uterus,
the surface is an external surface of the uterus.

15. The method of claim 11, wherein:
the inserting the instrument includes delivering to the target location an implant removably coupled to a distal end portion of the instrument.

16. The method of claim 11, wherein:
the inserting the head includes inserting a distal end portion of the reusable medical device into the body.

17. The method of claim 11, wherein:
the disposable tissue manipulation device includes a reservoir configured to (1) retain bodily particles received from the target location during any one of the inserting the head, manipulating the actuator, or the inserting the instrument, and (2) isolate the bodily particles within the reservoir from the reusable medical device, when the disposable tissue manipulation device is coupled to the reusable medial device.

18. The method of claim 11, wherein:
the inserting the instrument includes inserting the instrument after verification that the vacuum is above a predetermined threshold, the verification based on a vacuum gauge of the reusable medical device.

19. A kit, comprising:
a reusable medical device, the reusable medical device defining a vacuum chamber and an actuator at least partially disposed within the vacuum chamber such that movement of the actuator within the vacuum chamber produces a vacuum; and
a disposable assembly configured to be removably coupled to the reusable medical device, the disposable assembly including:
a head configured to be fluidically coupled to the vacuum chamber, the head defining a volume configured to at least partially circumscribe an opening to a bodily cavity associated with a target location, the head configured to exert the vacuum on a surface of the target location via the volume, and
a reservoir, the reservoir coupled to the head, the reservoir in fluid communication with the vacuum chamber of the reusable medical device when the reservoir is coupled to the reusable medical device, the reservoir including a filter configured to (1) limit passage therethrough of particles having a size above a threshold value, and (2) allow air to pass therethrough sufficient to maintain the vacuum within the disposable assembly.

20. The kit of claim 19, wherein the disposable assembly is a first disposable assembly, the head of the first disposable assembly is a first head, the first head having a first size, the kit further comprising:
a second disposable assembly configured to be removably coupled to the reusable medical device, the second disposable assembly having a second head, the second head having a second size different from the first size.

21. The kit of claim 19, further comprising:
a sterile packaging defining a sterile volume,
the disposable assembly disposed within the sterile volume of the sterile packaging.

22. The kit of claim 19, wherein:
the target location is a uterus.

23. The kit of claim 19, wherein:
the head includes a connection member configured to mate with a corresponding connection member of the reusable medical device such that the head can rotate relative to the reusable medical device with at least one degree of freedom.

24. The kit of claim 19, wherein:
the head includes a dilation member configured to transition between a first configuration and second configuration, the dilation member defining an opening when in the second configuration through which an insertion member can be moved when the head is coupled to reusable medical device.

25. The kit of claim 24, wherein:
the dilation member is tapered when in the first configuration, the dilation member configured to transition to the second configuration in response to a force received from the insertion member.

26. The kit of claim 19, wherein:
the head is configured to be removably coupled to a first portion of the reusable medical device via a first interference fit, the reservoir being configured to be removably coupled to a second portion of the reusable medical device via a second interference fit.

27. The kit of claim 19, wherein:
the reservoir defines a collection chamber configured to retain bodily fluid associated with the target location and received during a medical procedure, the filter of the reservoir being configured to isolate the bodily fluid within the collection chamber from the reusable medical device when the vacuum is (1) produced within the reusable medical device and (2) exerted on the surface associated with the target tissue during the medical procedure.

28. The kit of claim 19, wherein the reusable medical device includes a shaft defining a passageway configured to receive an insertion member, a portion of the shaft configured to be removably coupled to the head of the disposable assembly.

29. The kit of claim 28, further comprising:
the insertion member, a first portion of the insertion member configured to be disposed through the head of the disposable assembly and a second portion of the insertion member configured to be disposed within the bodily cavity, when the reusable medical device is coupled to the disposable assembly.

30. The kit of claim 19, wherein:
the reusable medical device includes a vacuum gauge configured to measure the vacuum produced within the vacuum chamber.
